(19) United States
(12) Patent Application Publication
(10) Pub. No.: US 2004/0235747 A1
(43) Pub. Date: Nov. 25, 2004

(54) Methods of treating and/or preventing autoimmune diseases

(75) Inventors: Illana Gozes, Ramat-Hasharon (IL); Daniel Offen, Kfar Haroeh (IL); Eliezer Giladi, Netania (IL); Eldad Melamed, Tel-Aviv (IL); Douglas E. Brenneman, North Wales, PA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: Ramot At Tel-Aviv University Ltd., Tel Aviv (IL); The Govt. of the USA as represented by the Secretary of the Dept. of Health & Human Services

(21) Appl. No.: 10/748,765

(22) Filed: Dec. 29, 2003

Related U.S. Application Data
(60) Provisional application No. 60/437,650, filed on Jan. 2, 2003.

(51) Int. Cl. A61K 38/10

(52) U.S. Cl. 514/14

(57) ABSTRACT

The present invention provides methods for preventing and/or treating autoimmune diseases, including multiple sclerosis, in a subject using ADNF polypeptides, by treating the subject with an Activity Dependent Neurotrophic Factor (ADNF) polypeptide.

### CROSS-REFERENCES TO RELATED APPLICATIONS

This application claims priority to U.S. Ser. No. 60/437,650, filed Jan. 2, 2003, herein incorporated by reference in its entirety.

This application is related to PCT WO 1/92333;U.S. Ser. No. 07/871,973 filed Apr. 22, 1992, now U.S. Pat. No. 5,767,240; U.S. Ser. No. 08/342, 297, filed Oct. 17, 1994 (published as WO96/11948), now U.S. Pat. No. 6,174,862; U.S. Ser. No. 60/037,404, filed Feb. 7, 1997 (published as WO98/35042); U.S. Ser. No. 09/187,330, filed Nov. 11, 1998 (published as WO00/27875); U.S. Ser. No. 09/267,511, filed Mar. 12, 1999 (published as WO00/53217); U.S. Ser. No. 60/149,956, filed Aug. 18, 1999 (published as WO01/12654); U.S. Ser. No. 60/208,944, filed May 31, 2000; and U.S. Ser. No. 60/267,805, filed Feb. 8, 2001; herein each incorporated by reference in their entirety.

### BACKGROUND OF THE INVENTION

Multiple sclerosis (MS) is a chronic disabling disease of the central nervous system (CNS), with relapsing-remitting or chronic-progressive clinical manifestations. The etiology of MS has not yet been fully elucidated, but it is believed that immunological mechanisms are involved in disease initiation and progression. Although the major histological hallmark of MS lesions in the CNS is demyelination with destruction of the myelin sheath and death of oligodendrocytes, it has been proposed that mild to moderate axonal damage and loss also occurs in the late chronic progressive stage of the disease. Pathological studies in MS patients have demonstrated a high frequency of terminal axonal damage correlating with irreversible neurological impairment.

At present, there are no known cures for MS and few effective treatments. The present invention addresses these and other problems.

### BRIEF SUMMARY OF THE INVENTION

Embodiments of the invention provide methods for preventing and/or treating autoimmune diseases in a subject by administering an Activity Dependent Neurotrophic Factor (ADNF) polypeptide in an amount sufficient to improve postnatal performance. The ADNF polypeptides include ADNF I and ADNF III (also referred to as ADNP) polypeptides, analogs, subsequences, and D-amino acid versions (either wholly D-amino acid peptides or mixed D- and L-amino acid peptides), and combinations thereof which contain their respective active core sites and provide neuroprotective and growth-promoting functions.

The ADNF I polypeptides have an active core site comprising the following amino acid sequence: Ser-Ala-Leu-Leu-Arg-Ser-Ile-Pro-Ala (“SALLRSIPA” or in short referred to as “SAL” or “ADNF-9”). The ADNF III polypeptides also have an active core site comprising a few amino acid residues, namely, the following amino acid sequence: Asn-Ala-Pro-Val-Ser-Ile-Pro-Gln (“NAPVSIPQ” or in short referred as “NAP”). These ADNF polypeptides have previously been shown, each on their own, to have remarkable potency and activity in animal models related to neurodegeneration.

In one embodiment, the method comprises administering an ADNF polypeptide, wherein the ADNF polypeptide is an ADNF I polypeptide comprising an active core site having the amino acid sequence of Ser-Ala-Leu-Leu-Arg-Ser-Ile-Pro-Ala (SEQ ID NO:1). In another embodiment, the method comprises administering a full length ADNF I polypeptide. In yet another embodiment, the method comprises administering an ADNF I polypeptide which consists of the amino acid sequence of Ser-Ala-Leu-Leu-Arg-Ser-Ile-Pro-Ala (SEQ ID NO:1). In yet another embodiment, the method comprises administering an ADNF I polypeptide, wherein the ADNF I polypeptide is selected from the group consisting of: Val-Leu-Gly-Gly-Gly-Ser-Ala-Leu-Leu-Arg-Ser-Ile-Pro-Ala (SEQ ID NO:14); Val-Glu-Glu-Gly-Ile-Val-Leu-Gly-Gly-Gly-Ser-Ala-Leu-Leu-Arg-Ser-Ile-Pro-Ala (SEQ ID NO:15); Leu-Gly-Gly-Gly-Ser-Ala-Leu-Leu-Arg-Ser-Ile-Pro-Ala (SEQ ID NO:16); Gly-Gly-Gly-Ser-Ala-Leu-Leu-Arg-Ser-Ile-Pro-Ala (SEQ ID NO:17); Gly-Gly-Ser-Ala-Leu-Leu-Arg-Ser-Ile-Pro-Ala (SEQ ID NO:18); and Gly-Ser-Ala-Leu-Leu-Arg-Ser-Ile-Pro-Ala (SEQ ID NO:19). In yet another embodiment, the method comprises administering an ADNF I polypeptide having up to about 20 amino acids at at least one of the N-terminus or the C-terminus of the active core site. In certain embodiments, the ADNF I polypeptide has up to 20 amino acids at both the N-terminus and the C-terminus of the ADNF I polypeptide.

In some embodiments, the method comprises administering an ADNF III polypeptide, wherein the ADNF polypeptide is a polypeptide comprising an active core site having the amino acid sequence of Asn-Ala-Pro-Val-Ser-Ile-Pro-Gln (SEQ ID NO:1). In yet another embodiment, the method comprises administering a full length ADNF III polypeptide. In yet another embodiment, the method comprises administering an ADNF I polypeptide which consists of the amino acid sequence of Asn-Ala-Pro-Val-Ser-Ile-Pro-Gln (SEQ ID NO:1). In yet another embodiment, the method comprises administering an ADNF III polypeptide, wherein the ADNF III polypeptide is selected from the group consisting of: Gly-Gly-Asn-Ala-Pro-Val-Ser-Ile-Pro-Gln (SEQ ID NO:2); Leu-Gly-Gly-Asn-Ala-Pro-Val-Ser-Ile-Pro-Gln-Gln-Ser (SEQ ID NO:3); Leu-Gly-Leu-Gly-Gly-Asn-Ala-Pro-Val-Ser-Ile-Pro-Gln-Gln-Ser (SEQ ID NO:4); and Ser-Val-Arg-Leu-Gly-Leu-Gly-Gly-Asn-Ala-Pro-Val-Ser-Ile-Pro-Gln-Gln-Ser (SEQ ID NO:5). In yet another embodiment, the method comprises administering an ADNF polypeptide having up to about 20 amino acids at at least one of the N-terminus and the C-terminus of the active core site. In certain embodiments, the ADNF polypeptide has up to 20 amino acids at both the N-terminus and the C-terminus of the ADNF polypeptide.

In yet another embodiment, the method comprises administering a mixture of an ADNF I polypeptide and an ADNF III polypeptide. Any one or more of the ADNF I polypeptides described herein can be mixed with any one or more of the ADNF III polypeptides described herein in this and other aspects of the invention.

In another embodiment, the active core site of the ADNF polypeptide comprises at least one D-amino acid. In another embodiment, the active core site of the ADNF polypeptide comprises all D-amino acids.

In yet another embodiment, at least one of the ADNF polypeptide is encoded by a nucleic acid that is administered to the subject.

In some embodiments, the subject has an autoimmune disease (e.g., multiple sclerosis). In some embodiments, the ADNF polypeptide is administered to prevent autoimmune disease (e.g., multiple sclerosis).

In some embodiments, the autoimmune disease is selected from the group consisting of multiple sclerosis, myasthenia gravis, Guillan-Barre syndrome (antiphospholipid syndrome), systemic lupus erytromatosis, Behcet's syndrome, Sjogrens syndrome, rheumatoid arthritis, Hashimoto's disease/hypothyroiditis, primary biliary cirrhosis, mixed connective tissue disease, chronic active hepatitis, Graves' disease/hyperthyroiditis, scleroderma, chronic idiopathic thrombocytopenic purpura, diabetic neuropathy and septic shock.

In some embodiments, the ADNF polypeptide is administered intranasally. In some embodiments, the ADNF polypeptide is administered orally. In some embodiments, the ADNF polypeptide is injected.

These and other aspects of the present invention will become apparent to those skilled in the art from the following detailed description of the invention, the accompanying drawings, and the appended claims.

### Definitions

The phrase “ADNF polypeptide” refers to one or more activity dependent neurotrophic factors (ADNF) that have an active core site comprising the amino acid sequence of SALLRSIPA (referred to as “SAL”) or NAPVSIPQ (referred to as “NAP”), or conservatively modified variants thereof that have neurotrophic/neuroprotective activity as measured with in vitro cortical neuron culture assays described by, e.g., Hill et al., Brain Res. 603, 222-233 (1993); Brenneman et al., Nature 335, 636 (1988); Brenneman et al., Dev. Brain Res. 51:63 (1990); Forsythe & Westbrook, J. Physiol. Lond. 396:515 (1988); Gozes et al., Proc. Natl. Acad. Sci. USA 93: 427 (1996). An ADNF polypeptide can be an ADNF I polypeptide, an ADNF III polypeptide, their alleles, polymorphic variants, analogs, interspecies homolog, any subsequences thereof (e.g., SALLRSIPA or NAPVSIPQ) or lipophilic variants that exhibit neuroprotective/neurotrophic action on, e.g., neurons originating in the central nervous system either in vitro or in vivo. An “ADNF polypeptide” can also refer to a mixture of an ADNF I polypeptide and an ADNF III polypeptide.

The term “ADNF I” refers to an activity dependent neurotrophic factor polypeptide having a molecular weight of about 14,000 Daltons with a pI of 8.3±0.25. As described above, ADNF I polypeptides have an active site comprising an amino acid sequence of Ser-Ala-Leu-Leu-Arg-Ser-Ile-Pro-Ala (also referred to as “SALLRSIPA” or “SAL” or “ADNF-9”). See, Brenneman & Gozes, J. Clin. Invest. 97:2299-2307 (1996); Glazner et al., Anat. Embryol. 200: 65 (1999); Brenneman et al., J. Pharm. Exp. Ther., 285:619-27 (1998); Gozes & Brenneman, J. Mol. Neurosci. 7:235-244 (1996); Gozes et al., Dev. Brain Res. 99:167-175 (1997); and Gozes Trends in Neurosci. 24: 700 (2001), all of which are herein incorporated by reference. Unless indicated as otherwise, “SAL” refers to a peptide having an amino acid sequence of Ser-Ala-Leu-Leu-Arg-Ser-Ile-Pro-Ala, not a peptide having an amino acid sequence of Ser-Ala-Leu. A full length amino acid sequence of ADNF I can be found in WO 96/11948, herein incorporated by reference in its entirety.

The phrase “ADNF III polypeptide” or “ADNF III” refers to one or more activity dependent neurotrophic factors (ADNF) that have an active core site comprising the amino acid sequence of NAPVSIPQ (referred to as “NAP”), or conservatively modified variants thereof that have neurotrophic/neuroprotective activity as measured with in vitro cortical neuron culture assays described by, e.g., Hill et al., Brain Res. 603, 222-233 (1993); Gozes et al., Proc. Natl. Acad. Sci. USA 93, 427-432 (1996). An ADNF polypeptide can be an ADNF III polypeptide, allelelic or polymorphic variant, analog, interspecies homolog, or any subsequences thereof (e.g., NAPVSIPQ) that exhibit neuroprotective/neurotrophic action on, e.g., neurons originating in the central nervous system either in vitro or in vivo. ADNF III polypeptides can range from about eight amino acids and can have, e.g., between 8-20, 8-50, 10-100 or about 1000 or more amino acids.

Full length human ADNF III has a predicted molecular weight of 123,562.8 Da (>1000 amino acid residues) and a pI of about 6.97. As described above, ADNF III polypeptides have an active site comprising an amino acid sequence of Asn-Ala-Pro-Val-Ser-Ile-Pro-Gln (also referred to as “NAPVSIPQ” or “NAP”). See, Zamostiano et al., J. Biol. Chem. 276:708-714 (2001) and Bassan et al., J. Neurochem. 72:1283-1293 (1999), each of which is incorporated herein by reference. Unless indicated as otherwise, “NAP” refers to a peptide having an amino acid sequence of Asn-Ala-Pro-Val-Ser-Ile-Pro-Gln, not a peptide having an amino acid sequence of Asn-Ala-Pro. Full-length sequences of ADNF III can be found in WO 98/35042 and WO 00/27875.

The term “subject” refers to any mammal, in particular human, at any stage of life.

The term “contacting” is used herein interchangeably with the following: combined with, added to, mixed with, passed over, incubated with, flowed over, etc. Moreover, the ADNF III polypeptides or nucleic acids encoding them of the present invention can be “administered” by any conventional method such as, for example, parenteral, oral, topical, and inhalation routes. In some embodiments, parenteral and nasal inhalation routes are employed.

“An amount sufficient,” “an effective amount” or “a therapeutically effective amount” is that amount of a given ADNF polypeptide that prevents the onset of symptoms of an autoimmune disease or that partially or completely reduces the symptoms of an autoimmune disease. For example, “an amount sufficient,” “an effective amount” or “a therapeutically effective amount” is that amount of a given ADNF polypeptide that decreases the frequency of myelin basic protein (MBP)-reactive cells in a subject or that reduces in TNF and IFN-α, or results in a delay in sustained progression of disability in a Kaplan-Meier curve, as described herein. The dosing range can vary depending on the ADNF polypeptide used, the route of administration and the potency of the particular ADNF polypeptide, but can readily be determined using the foregoing assays.

The terms “isolated,” “purified,” or “biologically pure” refer to material that is substantially or essentially free from components which normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. In particular, an isolated ADNF nucleic acid is separated from open reading frames that flank the ADNF gene and encode proteins other than ADNF. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

“Nucleic acid” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. The term nucleic acid is used interchangeably with gene, cDNA, mRNA, oligonucleotide, and polynucleotide.

The terms “polypeptide,” “peptide,” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers.

The term “amino acid” refers to naturally occurring amino acids, amino acid analogs, and amino acid mimetics that function in a manner similar to the naturally occurring and analog amino acids. Naturally-occurring amino acids are those encoded by the genetic code, as well as those amino acids that are later modified, e.g., hydroxyproline, γ-carboxyglutamate, and O-phosphoserine. Amino acid analogs refers to synthetic amino acids that have the same basic chemical structure as a naturally occurring amino acid, i.e., an a carbon that is bound to a hydrogen, a carboxyl group, an amino group, and an R group (e.g., homoserine, norleucine, methionine sulfoxide, methionine methyl sulfonium). Such analogs have modified R groups (e.g., norleucine) or modified peptide backbones, but retain the same basic chemical structure as a naturally occurring amino acid. Both naturally occurring and analog amino acids can be made synthetically. Amino acid mimetics refer to chemical compounds that have a structure that is different from the general chemical structure of an amino acid, but that function in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refer to those nucleic acids which encode identical or essentially identical amino acid sequences, or where the nucleic acid does not encode an amino acid sequence, to essentially identical sequences. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein that encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following groups each contain amino acids that are conservative substitutions for one another:

1) Alanine (A), Glycine (G);

2) Serine (S), Threonine (T);

3) Aspartic acid (D), Glutamic acid (E);

4) Asparagine (N), Glutamine (Q);

5) Cysteine (C), Methionine (M);

6) Arginine (R), Lysine (K), Histidine (H);

7) Isoleucine (I), Leucine (L), Valine (V); and

8) Phenylalanine (F), Tyrosine (Y), Tryptophan (W).

(see, e.g., Creighton, Proteins (1984)).

The terms “identical” or percent “identity,” in the context of two or more nucleic acids or polypeptide sequences, refer to two or more sequences or subsequences that are the same. The terms “substantially identical” refers to two or more nucleic acid or polypeptide sequences or subsequences that have a specified percentage of amino acid residues or nucleotides (i.e., 60%, 70%, 80%, 90%, 95% or 99% identity) that are the same, when compared and aligned for maximum correspondence over a comparison window, as measured using one of the following sequence comparison algorithms or by manual alignment and visual inspection. The present invention encompasses embodiments employing ADNF I or ADNF III polypeptides substantially identical to SEQ ID NO:1, SEQ ID NO:2, or full length human ADNF polypeptide. Preferably, the percent identity exists over a region of the sequence that is at least about 25 amino acids in length, more preferably over a region that is 50 or 100 amino acids in length or the entire polypeptide sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

An algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 are used, with the parameters described herein, to determine percent sequence identity for the nucleic acids and proteins of the invention. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

Another indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with a wash in 0.2×SSC, and 0.1% SDS at 65° C. The present invention encompasses nucleic acids that hybridize to polynucleotides encoding SEQ ID NO:1, SEQ ID NO:2, or other ADNF polypeptides exemplified herein or known to those of skill in the art.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency.

### BRIEF DESCRIPTION OF THE DRAWINGS

FIG. 1 illustrates the amount of autoimmune encephalomyelitis in mice treated with myelin-oligodendrocyte glycoprotein (MOG) and with or without treatment with NAP.

FIG. 2 illustrates the thymidine incorporation into splenocytes in a MOG-induced mouse model in the presence (filled circles) or absence (open circles) of NAP.

### DETAILED DESCRIPTION OF THE INVENTION

The present invention provides methods for preventing and treating an autoimmune disease in a subject. The method comprises administering to the subject an ADNF III polypeptide in an amount sufficient prevent or treat an autoimmune disease such as multiple sclerosis, myasthenia gravis, Guillan-Barre syndrome (antiphospholipid syndrome), systemic lupus erytromatosis, Behcet's syndrome, Sjogrens syndrome, rheumatoid arthritis, Hashimoto's disease/hypothyroiditis, primary biliary cirrhosis, mixed connective tissue disease, chronic active hepatitis, Graves' disease/hyperthyroiditis, scleroderma, chronic idiopathic thrombocytopenic purpura, diabetic neuropathy and septic shock.

I. ADNF Polypeptides

Any suitable ADNF polypeptides can be administered in embodiments of the invention. For example, an ADNF polypeptide can be an ADNF I polypeptide, an ADNF III polypeptide, or a mixture thereof. In some embodiments, ADNF polypeptides may comprise all L-amino acids, all D-amino acids, or a combination thereof. When ADNF polypeptides are to be orally administered, preferably an ADNF polypeptide comprises at least one D-amino acid within its active core site, more preferably at the N-terminus and/or the C-terminus of the active core site, and even more preferably at the entire active core site or over the length of the molecule. Alternatively, the D-amino acid can be at any suitable position in the polypeptide sequence. Since D-enatiomers of polypeptides are enzymatically more stable than their L-enatiomers, particularly in the gastrointestinal tract, an ADNF polypeptide comprising D-amino acids are particularly useful for oral administration.

In one aspect, the method comprises administering an ADNF I polypeptide that comprises an active core site having the following amino acid sequence: Ser-Ala-Leu-Leu-Arg-Ser-Ile-Pro-Ala. In one embodiment, the ADNF I polypeptide consists of an active core site that has an amino acid sequence of Ser-Ala-Leu-Leu-Arg-Ser-Ile-Pro-Ala. In another embodiment, the ADNF I polypeptide can comprise additional amino acids at the N-terminus and/or at the C-terminus of the active core site. For example, the ADNF I polypeptide can comprise up to 40 amino acids at the N-terminus and/or the C-terminus of the active core site. In another example, the ADNF I polypeptide can comprise up to 20 amino acids at the N-terminus and/or the C-terminus of the active core site. In yet another example, the ADNF I polypeptide can comprise up to 10 amino acids at the N-terminus and/or the C-terminus of the active core site. In yet another embodiment, the ADNF I polypeptide can be a full length ADNF I polypeptide.

In another aspect, the method comprises administering to the subject an ADNF III polypeptide that comprises an active core site having the following amino acid sequence: Asn-Ala-Pro-Val-Ser-Ile-Pro-Gln. In one embodiment, the ADNF III polypeptide consists of an active core site that has an amino acid sequence of Asn-Ala-Pro-Val-Ser-Ile-Pro-Gln. In another embodiment, the ADNF III polypeptide can comprise additional amino acids at the N-terminus and/or at the C-terminus of the active core site. For example, the ADNF III polypeptide can comprise up to 40 amino acids at the N-terminus and/or the C-terminus of the active core site. In another example, the ADNF III polypeptide can comprise up to 20 amino acids at the N-terminus and/or the C-terminus of the active core site. In yet another example, the ADNF III polypeptide can comprise up to 10 amino acids at the N-terminus and/or the C-terminus of the active core site. In yet another embodiment, the ADNF III polypeptide can be a full length ADNF III polypeptide.

In a preferred embodiment, the ADNF I polypeptide comprises an amino acid sequence of (R¹)ₓ—Ser-Ala-Leu-Leu-Arg-Ser-Ile-Pro-Ala-(R²)_{y}, and the ADNF III polypeptide comprises an amino acid sequence of (R³)_{w}-Asn-Ala-Pro-Val-Ser-Ile-Pro-Gln-(R⁴)_{z}.

In the above formula, each of R¹, R², R³, and R⁴, if present, is an amino acid sequence comprising from 1 to about 40 amino acids wherein each amino acid is independently selected. The term “independently selected” is used herein to indicate that the amino acids making up, for example, the amino acid sequence R¹ may be identical or different (e.g., all of the amino acids in the amino acid sequence may be threonine, etc.). Moreover, as previously explained, the amino acids making up the amino acid sequence R may be either naturally occurring amino acids, or known analogues of natural amino acids that functions in a manner similar to the naturally occurring amino acids (i.e., amino acid mimetics). This discussion pertaining to R¹ is fully applicable to R², R³, and R⁴.

Within the above formula for the ADNF I polypeptide, x and y are independently selected and are equal to zero or one. The term independently selected is used herein to indicate that x and y may be identical or different. For example, x and y may both be zero or, alternatively, x and y may both be one. In addition, x may be zero and y may be one or, alternatively, x may be one and y may be zero. Moreover, if x and y are both one, the amino acid sequences R¹ and R² may be the same or different. As such, the amino acid sequences R¹ and R² are independently selected. If R¹ and R² are the same, they are identical in terms of both chain length and amino acid composition. For example, both R¹ and R² may be Val-Leu-Gly-Gly-Gly. If R¹ and R² are different, they can differ from one another in terms of chain length and/or amino acid composition and/or order of amino acids in the amino acids sequences. For example, R¹ may be Val-Leu-Gly-Gly-Gly, whereas R² may be Val-Leu-Gly-Gly. Alternatively, R¹ may be Val-Leu-Gly-Gly-Gly, whereas R² may be Val-Leu-Gly-Gly-Val. Alternatively, R¹ may be Val-Leu-Gly-Gly-Gly, whereas R² may be Gly-Val-Leu-Gly-Gly.

Similarly, w and z are independently selected and are equal to zero or one within the above formula for the ADNF III polypeptide. The term independently selected is used herein to indicate that w and z may be identical or different. For example, w and z may both be zero or, alternatively, w and z may both be one. In addition, w may be zero and z may be one or, alternatively, w may be one and z may be zero. Moreover, if w and z are both one, the amino acid sequences R³ and R⁴ may be the same or different. As such, the amino acid sequences R³ and R⁴ are independently selected. If R³ and R⁴ are the same, they are identical in terms of both chain length and amino acid composition. For example, both R³ and R⁴ may be Leu-Gly-Leu-Gly-Gly. If R³ and R⁴ are different, they can differ from one another in terms of chain length and/or amino acid composition and/or order of amino acids in the amino acids sequences. For example, R³ may be Leu-Gly-Leu-Gly-Gly, whereas R⁴ may be Leu-Gly-Leu-Gly. Alternatively, R³ may be Leu-Gly-Leu-Gly-Gly, whereas R⁴ may be Leu-Gly-Leu-Gly-Leu.

Within the scope, certain ADNF I and ADNF III polypeptides are preferred, namely those in which x, y, w, and z are all zero (i.e., SALLRSIPA and NAPVSIPQ, respectively). Equally preferred are ADNF I polypeptides in which x is one; R¹ is Val-Leu-Gly-Gly-Gly; and y is zero. Also equally preferred are ADNF I polypeptides in which x is one; R¹ is Val-Glu-Glu-Gly-Ile-Val-Leu-Gly-Gly-Gly; and y is zero. Also equally preferred are ADNF III polypeptides in which w is one; R³ is Gly-Gly; and z is zero. Also equally preferred are ADNF III polypeptides in which w is one; R³ is Leu-Gly-Gly; z is one; and R⁴ is Gln-Ser. Also equally preferred are ADNF III polypeptides in which w is one; R³ is Leu-Gly-Leu-Gly-Gly-; z is one; and R⁴ is Gln-Ser. Also equally preferred are ADNF III polypeptides in which w is one; R³ is Ser-Val-Arg-Leu-Gly-Leu-Gly-Gly; z is one; and R⁴ is Gln-Ser. Additional amino acids can be added to both the N-terminus and the C-terminus of these active sites (SALLRSIPA or NAPVSIPQ) without loss of biological activity as evidenced by the fact that the intact ADNF I or ADNF III growth factors exhibit extraordinary biological activity. See, U.S. Ser. No. 08/324,297, filed Oct. 17, 1994 (also published as WO96/11948) for the description of ADNF I polypeptides; and U.S. Ser. No. 60/037,404 filed Feb. 27, 1997 and U.S. Ser. No. 60/059,621 filed, Sep. 23, 1997 (also published as WO98/35042) for the description of ADNF III polypeptides, all of which are incorporated herein by reference.

In yet another aspect, the method comprises administering to the subject a mixture of an ADNF I polypeptide and an ADNF III polypeptide. Any one or more of the ADNF I polypeptides described herein can be mixed with any one or more of the ADNF III polypeptides described herein. A mixture of an ADNF I polypeptide and an ADNF III polypeptide can be a blend of two or more of these polypeptides. A mixture of an ADNF I polypeptide and an ADNF III polypeptide can also refer to one or more of ADNF I polypeptides that are coupled (directly or indirectly) to one or more of ADNF III polypeptides. For example, an ADNF I polypeptide can be covalently linked to an ADNF III polypeptide. A mixture of ADNF I polypeptides and ADNF III polypeptides can be prepared as a single composition and can be administered to a subject. Alternatively, an ADNF I polypeptide and an ADNF III polypeptide can be prepared as separate compositions. The separate compositions can then be administered simultaneously or sequentially to the subject. Furthermore, different proportions of an ADNF I polypeptide and an ADNF III polypeptide can be administered to a subject. For example, the subject can be administered with ADNF polypeptides, wherein the ratio of an ADNF I polypeptide and an ADNF III polypeptide can be in the range of 1:100 to 100:1, 1:10 to 10:1, or 1:2 to 2:1.

In some aspects, the ADNF polypeptides are linked to a PEG, lipid, or other molecule known in the art so as to make the polypeptide lipophilic. In some aspects, the ADNF polypeptides are in a liposome.

In yet another aspect, other ADNF polypeptide (including their alleles, polymorphic variants, species homologs and subsequences thereof) can be used to prevent or treat an autoimmune disease. Autoimmune diseases are well-known. See, e.g., HARRISON'S PRINCIPLES OF INTERNAL MEDICINE (eds., Fauci, et al., 1998). Exemplary autoimmune diseases include, e.g., multiple sclerosis, myasthenia gravis, Guillan-Barre syndrome (antiphospholipid syndrome), systemic lupus erytromatosis, Behcet's syndrome, Sjogrens syndrome, rheurnatoid arthritis, Hashimoto's disease/hypothyroiditis, primary biliary cirrhosis, mixed connective tissue disease, chronic active hepatitis, Graves' disease/hyperthyroiditis, scleroderma, chronic idiopathic thrombocytopenic purpura, diabetic neuropathy and septic shock.

II. Administration and Pharmaceutical Compositions

ADNF polypeptides and nucleic acids encoding ADNF polypeptides can be administered to a subject using any suitable methods known in the art. See, e.g., Gozes, et al., Trends in Neuroscience, 24(12):700-705 (2001); Gozes, et al., J. Molec. Neurosci. 19:167-170 (2002); Leker, et al., Stroke. 33(4):1085-1092 (2002); Gozes, et al., “Intranasal delivery of bioactive peptides or peptide analogues enhances spatial memory and protects against cholinergic deficits” In: The Proceedings of the 44th Oholo Conference: The Blood Brain Barrier Drug Delivery and Brain Pathology. 363-370. For example, ADNF polypeptides or nucleic acids can be formulated as pharmaceutical compositions with a pharmaceutically acceptable diluent, carrier or excipient. Suitable formulations for use in the present invention are found in Remington's Pharmaceutical Sciences (17th ed. 1985)), which is incorporated herein by reference. A brief review of methods for drug delivery is also described in, e.g., Langer, Science 249:1527-1533 (1990), which is incorporated herein by reference. In addition, the pharmaceutical compositions comprising peptides and proteins are described in, e.g., Therapeutic Peptides and Proteins Formulations, Processing, and Delivery Systems, by Banga, Technomic Publishing Company, Inc., Lancaster, Pa. (1995).

ADNF polypeptides can be administered in any pharmaceutically acceptable composition. A pharmaceutically acceptable nontoxic composition is formed by incorporating any of normally employed excipients, and generally 10-95% of active ingredient and more preferably at a concentration of 25%-75%. Furthermore, to improve oral absorption of ADNF polypeptides, various carrier systems, such as nanoparticles, microparticles, liposomes, phospholipids, emulsions, erythrocytes, etc. can be used. The oral agents comprising ADNF polypeptides of the invention can be in any suitable form for oral administration, such as liquid, tablets, capsules, or the like. The oral formulations can be further coated or treated to prevent or reduce dissolution in stomach. See, e.g., Therapeutic Peptides and Proteins, Formulation, Processing, and Delivery Systems, by A. K. Banga, Technomic Publishing Company, Inc., 1995.

Furthermore, the ADNF polypeptides can be formulated for parenteral, topical, nasal, sublingual, gavage, or local administration. For example, the pharmaceutical compositions are administered parenterally, e.g., intravenously, subcutaneously, intradermally, or intramuscularly, or intranasally. Thus, the invention provides compositions for parenteral administration that comprise a solution of a single or mixture of ADNF polypeptides, dissolved or suspended in an acceptable carrier, preferably an aqueous carrier. A variety of aqueous carriers may be used including, for example, water, buffered water, 0.4% saline, 0.3% glycine, hyaluronic acid and the like. These compositions may be sterilized by conventional, well known sterilization techniques, or they may be sterile filtered. The resulting aqueous solutions may be packaged for use as is or lyophilized, the lyophilized preparation being combined with a sterile solution prior to administration. The compositions may contain pharmaceutically acceptable auxiliary substances as required to approximate physiological conditions including pH adjusting and buffering agents, tonicity adjusting agents, wetting agents and the like, such as, for example, sodium acetate, sodium lactate, sodium chloride, potassium chloride, calcium chloride, sorbitan monolaurate, triethanolamine oleate, etc. In one embodiment, a nucleic acid encoding an ADNF polypeptide is administered as a naked DNA.

For aerosol administration, ADNF polypeptides are preferably supplied in finely divided form along with a surfactant and propellant. The surfactant must, of course, be nontoxic, and preferably soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. A carrier can also be included, as desired, as with, e.g., lecithin for intranasal delivery.

For solid compositions, conventional nontoxic solid carriers may be used. Solid carriers include, for example, pharmaceutical grades of mannitol, lactose, starch, magnesium stearate, sodium saccharin, talcum, cellulose, glucose, sucrose, magnesium carbonate, and the like.

Small polypeptides including SALLRSIPA and NAPVSIPQ cross the blood brain barrier. For longer polypeptides that do not the cross blood brain barrier, methods of administering proteins to the brain are well known. For example, proteins, polypeptides, other compounds and cells can be delivered to the mammalian brain via intracerebroventricular (ICV) injection or via a cannula (see, e.g., Motta & Martini, Proc. Soc. Exp. Biol. Med. 168:62-64 (1981); Peterson et al., Biochem. Pharamacol. 31:2807-2810 (1982); Rzepczynski et al., Metab. Brain Dis. 3:211-216 (1988); Leibowitz et al., Brain Res. Bull. 21:905-912 (1988); Sramka et al., Stereotact. Funct. Neurosurg. 58:79-83 (1992); Peng et al., Brain Res. 632:57-67 (1993); Chem et al., Exp. Neurol. 125:72-81 (1994); Nikkhah et al., Neuroscience 63:57-72 (1994); Anderson et al., J. Comp. Neurol. 357:296-317 (1995); and Brecknell & Fawcett, Exp. Neurol. 138:338-344 (1996)). In particular, cannulas can be used to administer neurotrophic factors to mammals (see, e.g., Motta & Martini, Proc. Soc. Exp. Biol. Med. 168:62-64 (1981) (neurotensin); Peng et al., Brain Res. 632:57-67 (1993) (NGF); Anderson et al., J. Comp. Neurol. 357:296-317 (1995) (BDNF, NGF, neurotrophin-3).

Alternatively, longer ADNF polypeptides that do not cross blood brain barrier can be coupled with a material which assists the ADNF polypeptide to cross the blood brain barrier and to traverse the plasma membrane of a cell, or the membrane of an intra-cellular compartment such as the nucleus. Cellular membranes are composed of lipid-protein bilayers that are freely permeable to small, nonionic lipophilic compounds and are inherently impermeable to polar compounds, macromolecules, and therapeutic or diagnostic agents. However, proteins and other compounds such as liposomes have been described, which have the ability to translocate polypeptides such as ADNF polypeptides across a cell membrane.

For example, “membrane translocation polypeptides” have amphiphilic or hydrophobic amino acid subsequences that have the ability to act as membrane-translocating carriers. In one embodiment, homeodomain proteins have the ability to translocate across cell membranes. The shortest internalizable peptide of a homeodomain protein, Antennapedia, was found to be the third helix of the protein, from amino acid position 43 to 58 (see, e.g., Prochiantz, Current Opinion in Neurobiology 6:629-634 (1996)). Another subsequence, the hydrophobic domain of signal peptides, was found to have similar cell membrane translocation characteristics (see, e.g., Lin et al., J. Biol. Chem. 270:1 4255-14258 (1995)).

Examples of peptide sequences which can be linked to a ADNF polypeptide of the invention, for facilitating uptake of ADNF polypeptides into cells, include, but are not limited to: an 11 amino acid peptide of the tat protein of HIV (see Schwarze et al., Science 285:1569-1572 (1999)); a 20 residue peptide sequence which corresponds to amino acids 84-103 of the p16 protein (see Fahraeus et al., Current Biology 6:84 (1996)); the third helix of the 60-amino acid long homeodomain of Antennapedia (Derossi et al., J. Biol. Chem. 269:10444 (1994)); the h region of a signal peptide such as the Kaposi fibroblast growth factor (K-FGF) h region (Lin et al., supra); or the VP22 translocation domain from HSV (Elliot & O Hare, Cell 88:223-233 (1997)). Other suitable chemical moieties that provide enhanced cellular uptake may also be chemically linked to ADNF polypeptides.

Toxin molecules also have the ability to transport polypeptides across cell membranes. Often, such molecules are composed of at least two parts (called “binary toxins”): a translocation or binding domain or polypeptide and a separate toxin domain or polypeptide. Typically, the translocation domain or polypeptide binds to a cellular receptor, and then the toxin is transported into the cell. Several bacterial toxins, including Clostridium perfringens iota toxin, diphtheria toxin (DT), Pseudomonas exotoxin A (PE), pertussis toxin (PT), Bacillus anthracis toxin, and pertussis adenylate cyclase (CYA), have been used in attempts to deliver peptides to the cell cytosol as internal or amino-terminal fusions (Arora et al., J. Biol. Chem., 268:3334-3341 (1993); Perelle et al., Infect. Immun., 61:5147-5156 (1993); Stenmark et al., J. Cell Biol. 113:1025-1032 (1991); Donnelly et al., Proc. Nat'l Acad. Sci. USA 90:3530-3534 (1993); Carbonetti et al., Abstr. Annu. Meet. Am. Soc. Microbiol. 95:295 (1995); Sebo et al., Infect. Immun. 63:3851-3857 (1995); Klimpel et al., Proc. Nat'l Acad. Sci. USA 89:10277-10281 (1992); and Novak et al., J. Biol. Chem. 267:17186-17193 1992)).

Such subsequences can be used to translocate ADNF polypeptides across a cell membrane. ADNF polypeptides can be conveniently fused to or derivatized with such sequences. Typically, the translocation sequence is provided as part of a fusion protein. Optionally, a linker can be used to link the ADNF polypeptides and the translocation sequence. Any suitable linker can be used, e.g., a peptide linker.

The ADNF polypeptides and nucleic acids encoding ADNF polypeptides can also be introduced into an animal cell, preferably a mammalian cell, via a liposomes and liposome derivatives such as immunoliposomes and lipid:nucleic acid complexes. The term “liposome” refers to vesicles comprised of one or more concentrically ordered lipid bilayers, which encapsulate an aqueous phase. The aqueous phase typically contains the compound to be delivered to the cell, i.e., an ADNF polypeptide.

The liposome fuses with the plasma membrane, thereby releasing the ADNF polypeptides into the cytosol. Alternatively, the liposome is phagocytosed or taken up by the cell in a transport vesicle. Once in the endosome or phagosome, the liposome either degrades or fuses with the membrane of the transport vesicle and releases its contents.

In current methods of drug delivery via liposomes, the liposome ultimately becomes permeable and releases the encapsulated compound (in this case, an ADNF III polypeptide) at the target tissue or cell. For systemic or tissue specific delivery, this can be accomplished, for example, in a passive manner wherein the liposome bilayer degrades over time through the action of various agents in the body. Alternatively, active drug release involves using an agent to induce a permeability change in the liposome vesicle. Liposome membranes can be constructed so that they become destabilized when the environment becomes acidic near the liposome membrane (see, e.g., Proc. Nat'l Acad. Sci. USA 84:7851 (1987); Biochemistry 28:908 (1989)). When liposomes are endocytosed by a target cell, for example, they become destabilized and release their contents. This destabilization is termed fusogenesis. Dioleoylphosphatidylethanolamine (DOPE) is the basis of many “fusogenic” systems.

Such liposomes typically comprise an ADNF polypeptide and a lipid component, e.g., a neutral and/or cationic lipid, optionally including a receptor-recognition molecule such as an antibody that binds to a predetermined cell surface receptor or ligand (e.g., an antigen). A variety of methods are available for preparing liposomes as described in, e.g., Szoka et al., Ann. Rev. Biophys. Bioeng. 9:467 (1980), U.S. Pat. Nos. 4,186,183, 4,217,344, 4,235,871, 4,261,975, 4,485,054, 4,501,728, 4,774,085, 4,837,028, 4,235,871, 4,261,975, 4,485,054, 4,501,728, 4,774,085, 4,837,028, 4,946,787, PCT Publication No. WO 91/17424, Deamer & Bangham, Biochim. Biophys. Acta 443:629-634 (1976); Fraley, et al., Proc. Nat'l Acad. Sci. USA 76:3348-3352 (1979); Hope et al., Biochim. Biophys. Acta 812:55-65 (1985); Mayer et al., Biochim. Biophys. Acta 858:161-168 (1986); Williams et al., Proc. Nat'l Acad. Sci. USA 85:242-246 (1988); Liposomes (Ostro (ed.), 1983, Chapter 1); Hope et al., Chem. Phys. Lip. 40:89 (1986); Gregoriadis, Liposome Technology (1984) and Lasic, Liposomes: from Physics to Applications (1993)). Suitable methods include, for example, sonication, extrusion, high pressure/homogenization, microfluidization, detergent dialysis, calcium-induced fusion of small liposome vesicles and ether-fusion methods, all of which are well known in the art.

In certain embodiments of the present invention, it is desirable to target the liposomes of the invention using targeting moieties that are specific to a particular cell type, tissue, and the like. Targeting of liposomes using a variety of targeting moieties (e.g., ligands, receptors, and monoclonal antibodies) has been previously described (see, e.g., U.S. Pat. Nos. 4,957,773 and 4,603,044). Standard methods for coupling targeting agents to liposomes can be used. These methods generally involve incorporation into liposomes lipid components, e.g., phosphatidylethanolamine, which can be activated for attachment of targeting agents, or derivatized lipophilic compounds, such as lipid derivatized bleomycin. Antibody targeted liposomes can be constructed using, for instance, liposomes which incorporate protein A (see Renneisen et al., J. Biol. Chem., 265:16337-16342 (1990) and Leonetti et al., Proc. Nat'l Acad. Sci. USA 87:2448-2451 (1990).

Alternatively, nucleic acids encoding ADNF can also be used to provide a therapeutic dose of ADNF polypeptides. These nucleic acids can be inserted into any of a number of well-known vectors for the transfection of target cells and organisms. For example, nucleic acids are delivered as DNA plasmids, naked nucleic acid, and nucleic acid complexed with a delivery vehicle such as a liposome. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11: 162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Böhm (eds) (1995); and Yu et al., Gene Therapy 1: 13-26 (1994).

Methods of non-viral delivery of nucleic acids include lipofection, microinjection, biolistics, virosomes, liposomes, immunoliposomes, polycation or lipid:nucleic acid conjugates, naked DNA, artificial virions, and agent-enhanced uptake of DNA. Lipofection is described in, e.g., U.S. Pat. No. 5,049,386, U.S. Pat. No. 4,946,787; and U.S. Pat. No. 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424, WO 91/16024. Delivery can be to cells (ex vivo administration) or target tissues (in vivo administration).

In some therapeutic applications, a mixture of ADNF I and ADNF III polypeptides of the invention is administered to a patient in an amount sufficient to decrease symptoms of an autoimmune disease. An amount adequate to accomplish this is defined as “therapeutically effective dose.” Amounts effective for this use will depend on, for example, the particular ADNF I or ADNF III polypeptide employed, the manner of administration, the weight and general state of health of the patient, and the judgment of the prescribing physician. “Therapeutically effective dose” also encompasses doses that are sufficient to prevent an autoimmune disease from developing in a subject. Thus, prophylactic doses are encompassed by the term “therapeutically effective dose.”

III. Identification of Candidates for Multiple Sclerosis Treatment and Prevention

Some patients suitable for treatment with ADNF III polypeptides may be identified by criteria establishing a diagnosis of clinically definite multiple sclerosis (MS) as defined by the workshop on the diagnosis of MS (Poser et al., Ann. Neurol. 13:227 (1983)). Briefly, an individual with clinically definite MS has had one attack and clinical evidence of either lesions or clinical evidence of one lesion and paraclinical evidence of another, separate lesion. Definite MS may also be diagnosed by evidence of an attack and oligoclonal bands of IgG in cerebrospinal fluid or by combination of an attack, clinical evidence of two lesions and oligoclonal band of IgG in cerebrospinal fluid. Slightly lower criteria are used for a diagnosis of clinically probable MS.

Effective treatment of multiple sclerosis may be examined in several different ways. Satisfying any of the following criteria evidences effective treatment. Three main criteria are used: EDSS (extended disability status scale), appearance of exacerbations or MRI (magnetic resonance imaging).

The EDSS is a means to grade clinical impairment due to MS (Kurtzke, Neurology 33:1444 (1983)). Eight functional systems are evaluated for the type and severity of neurologic impairment. Briefly, prior to treatment, patients are evaluated for impairment in the following systems: pyramidal, cerebella, brainstem, sensory, bowel and bladder, visual, cerebral, and other. Follow-ups are conducted at defined intervals. The scale ranges from 0 (normal) to 10 (death due to MS). In some embodiments, a decrease of at least one full step represents an effective treatment in the context of the present invention (Kurtzke, Ann. Neurol. 36:573-79 (1994)).

Exacerbations are defined as the appearance of a new symptom that is attributable to MS and accompanied by an appropriate new neurologic abnormality (IFNB MS Study Group, supra). In addition, the exacerbation must last at least 24 hours and be preceded by stability or improvement for at least 30 days. Briefly, patients are given a standard neurological examination by clinicians. Exacerbations are either mild, moderate, or severe according to changes in a Neurological Rating Scale (Sipe et al., Neurology 34:1368 (1984)). An annual exacerbation rate and proportion of exacerbation-free patients are determined. In some embodiments, therapy is effective if there is a statistically significant difference in the rate or proportion of exacerbation-free patients between the treated group and the placebo group (or for a single subject, after treatment with an ADNF III polypeptide compared to before the subject was treated) for either of these measurements. In addition, time to first exacerbation and exacerbation duration and severity may also be measured. In some embodiments, a measure of effectiveness using an ADNF III polypeptide in this regard is a statistically significant difference in the time to first exacerbation or duration and severity in the treated group compared to a control group.

MRI can be used to measure active lesions using gadolinium-DTPA-enhanced imaging (McDonald et al. Ann. Neurol. 36:14, 1994) or the location and extent of lesions using T₂-weighted techniques. Briefly, baseline MRIs are obtained. The same imaging plane and patient position are used for each subsequent study. Positioning and imaging sequences are chosen to maximize lesion detection and facilitate lesion tracing. The same positioning and imaging sequences are used on subsequent studies. The presence, location, and extent of MS lesions are determined by radiologists. Areas of lesions are outlined and summed slice by slice for total lesion area. At least three aspects can be examined: evidence of new lesions, rate of appearance of active lesions, percentage change in lesion area (see, e.g., Paty et al., Neurology 43:665, 1993). In some embodiments, improvement due to administration of ADNF III polypeptides can be established when there is a statistically significant improvement in an individual patient compared to baseline or in a treated group versus a placebo group.

Candidate patients for prevention of multiple sclerosis may be identified by the presence of genetic factors. For example, a majority of MS patients have HLA-type DR2a and DR2b. The MS patients having genetic dispositions to MS who are suitable for treatment fall within two groups. The first group includes patients with early disease of the relapsing remitting type. Entry criteria would include disease duration of more than one year, EDSS score of 1.0 to 3.5, exacerbation rate of more than 0.5 per year, and free of clinical exacerbations for 2 months prior to study. The second group would include people with disease progression greater than 1.0 EDSS unit/year over the past two years.

Efficacy of the peptide analogue in the context of prevention is judged based on the following criteria: frequency of myelin basic protein (MBP)-reactive T-cells determined by limiting dilution, proliferation response of MBP-reactive T-cell lines and clones, and cytokine profiles of T-cell lines and clones to MBP established from patients. Effective doses can decrease the frequency of reactive cells, reduce proliferation of MBP-reactive T-cells, and/or reduce levels of TNF and IFN-α. Clinical measurements include the relapse rate in one and two year intervals, and a change in EDSS, including time to progression from baseline of 1.0 unit on the EDSS which persists for six months. On a Kaplan-Meier curve, a delay in sustained progression of disability shows efficacy. Other criteria include a change in area and volume of T2 images on MRI, and the number and volume of lesions determined by gadolinium enhanced images. In some embodiments, if any one or more of these parameters are changed in a subject treated with ADNF III polypeptides by, e.g., about 10%, optionally at least about 20%, at least about 30%, at least about 40%, at least about 50%, at least about 60%, at least about 70%, at least about 80%, at least about 90% or up to about 100% compared to before treatment, then the ADNF III polypeptides are therapeutically effective.

IV. Methods for Production of ADNF Polypeptides

A. Recombinant Methods for Production of ADNF Polypeptides

i. Cloning and Isolation of ADNF Nucleic Acids

Several specific nucleic acids encoding ADNF polypeptides are described herein. See, also, e.g., Zamostiano et al., J. Biol. Chem. 276:708-714 (2001), and Bassan et al., J. Neurochem 72:1283-1293 (1999), the teachings of which are hereby incorporated in their entirety by reference. These nucleic acids can be made using standard recombinant or synthetic techniques. Given the nucleic acids of the present invention, one of skill can construct a variety of clones containing functionally equivalent nucleic acids, such as nucleic acids that encode the same ADNF polypeptides. Cloning methodologies to accomplish these ends, and sequencing methods to verify the sequence of nucleic acids are well known in the art. Examples of appropriate cloning and sequencing techniques, and instructions sufficient to direct persons of skill through many cloning exercises are found in Sambrook et al., Molecular Cloning—A Laboratory Manual (2nd ed. 1989) and Current Protocols in Molecular Biology (Ausubel et al., eds., 1994).

In addition, product information from manufacturers of biological reagents and experimental equipment also provide information useful in known biological methods. Such manufacturers include the SIGMA chemical company (Saint Louis, Mo.), R&D systems (Minneapolis, Minn.), Pharmacia LKB Biotechnology (Piscataway, N.J.), CLONTECH Laboratories, Inc. (Palo Alto, Calif.), Chem Genes Corp., Aldrich Chemical Company (Milwaukee, Wis.), Glen Research, Inc., GIBCO BRL Life Technologies, Inc. (Gaithersberg, Md.), Fluka Chemica-Biochemika Analytika (Fluka Chemie AG, Buchs, Switzerland), Invitrogen (San Diego, Calif.), and Applied Biosystems (Foster City, Calif.), as well as many other commercial sources known to one of skill.

The nucleic acid compositions of this invention, whether RNA, cDNA, genomic DNA or a hybrid of the various mixtures, are isolated from biological sources, such as astrocyte, neuroblastoma cells, or fibroblasts, or synthesized in vitro. The nucleic acids of the invention are present in transformed or transfected cells, in transformed or transfected cell lysates, or in a partially purified or substantially pure form.

In vitro amplification techniques suitable for amplifying sequences for use as molecular probes or generating nucleic acid fragments for subsequent subcloning are known. Examples of techniques sufficient to direct persons of skill through such in vitro amplification methods, including the polymerase chain reaction (PCR), the ligase chain reaction (LCR), Qβ-replicase amplification and other RNA polymerase mediated techniques (e.g., NASBA), are found in Berger, Sambrook et al. and Ausubel et al., all supra, as well as in U.S. Pat. No. 4,683,202; PCR Protocols A Guide to Methods and Applications (Innis et al., eds., 1990); Arnheim & Levinson (Oct. 1, 1990) C&EN 36-47; The Journal Of NIH Research 3:81-94 (1991); Kwoh et al., Proc. Natl. Acad. Sci. USA 86:1173 (1989); Guatelli et al., Proc. Natl. Acad. Sci. USA 87:1874 (1990); Lomell et al., J. Clin. Chem 35:1826 (1989); Landegren et al., Science 241:1077-1080 (1988); Van Brunt, Biotechnology 8:291-294 (1990); Wu & Wallace, Gene 4:560 (1989); Barringer et al., Gene 89:117 (1990); and Sooknanan & Malek, Biotechnology 13:563-564 (1995). Improved methods of cloning in vitro amplified nucleic acids are described in U.S. Pat. No. 5,426,039. Improved methods of amplifying large nucleic acids are summarized in Cheng et al., Nature 369:684-685 (1994) and the references cited therein. One of skill will appreciate that essentially any RNA can be converted into a double stranded DNA suitable for restriction digestion, PCR expansion and sequencing using reverse transcriptase and a polymerase.

Oligonucleotides for use as probes, for example, with in vitro ADNF nucleic acid amplification methods, or for use as nucleic acid probes to detect ADNF nucleic acids, are typically synthesized chemically according to the solid phase phosphoramidite triester method described by Beaucage & Caruthers, Tetrahedron Letts. 22(20):1859-1862 (1981), e.g., using an automated synthesizer, e.g., as described in Needham-VanDevanter et al., Nucleic Acids Res. 12:6159-6168 (1984). Oligonucleotides can also be custom made and ordered from a variety of commercial sources known to those of skill in the art. Purification of oligonucleotides, where necessary, is typically performed by either native acrylamide gel electrophoresis, or by anion-exchange HPLC as described in Pearson & Regnier, J. Chrom. 255:137-149 (1983). The sequence of the synthetic oligonucleotides can be verified using the chemical degradation method of Maxam & Gilbert, in Methods in Enzymology 65:499-560 (Grossman & Moldave, eds., 1980).

One of skill will recognize many ways of generating alterations in a given nucleic acid sequence. Such well-known methods include site-directed mutagenesis, PCR amplification using degenerate oligonucleotides, exposure of cells containing the nucleic acid to mutagenic agents or radiation, chemical synthesis of a desired oligonucleotide (e.g., in conjunction with ligation and/or cloning to generate large nucleic acids) and other well-known techniques (see, Giliman & Smith, Gene 8:81-97 (1979); Roberts et al., Nature 328:731-734 (1987); and Sambrook et al., Molecular Cloning—A Laboratory Manual (2nd ed. 1989)).

ii. Recombinant Expression of ADNF III Polypeptides

In one embodiment, the polypeptides, or subsequences thereof, are synthesized using recombinant nucleic acid methodology. Generally, this involves creating a nucleic acid sequence that encodes the protein, placing the nucleic acid in an expression cassette under the control of a particular promoter, expressing the protein in a host cell, isolating the expressed protein and, if required, renaturing the protein.

Once a nucleic acid encoding an ADNF polypeptide of the invention is isolated and cloned, the nucleic acid is optionally expressed in recombinantly engineered cells known to those of skill in the art. Examples of such cells include, but are not limited to, bacteria, yeast, plant, filamentous fungi, insect (especially employing baculoviral vectors) and mammalian cells. The recombinant nucleic acids are operably linked to appropriate control sequences for expression in the selected host. For E. coli, example control sequences include the T7, trp, or lambda promoters, a ribosome binding site and, preferably, a transcription termination signal. For eukaryotic cells, the control sequences typically include a promoter and, preferably, an enhancer derived from immunoglobulin genes, SV40, cytomegalovirus, etc., and a polyadenylation sequence, and may include splice donor and acceptor sequences.

If desired, recombinant nucleic acids can be constructed to encode a fusion polypeptide comprising an ADNF polypeptide. In one example, a nucleic acid encoding an ADNF polypeptide (e.g., an ADNF II polypeptide, an ADNF III polypeptide, a fusion ADNF I/ADNF III polypeptide, etc.) can be linked with another nucleic acid, such as a portion of HIV tat nucleic acid, which facilitates the delivery of the ADNF polypeptide into tissues. In yet another example, a nucleic acid encoding an ADNF polypeptide can be linked to nucleic acids that encode affinity tags to facilitate protein purification protocol. An ADNF nucleic acid and a heterologous polynucleotide sequence can be modified to facilitate their fusion and subsequent expression of fusion polypeptides. For example, the 3′ stop codon of the ADNF polynucleotide sequence can be substituted with an in frame linker sequence, which may provide restriction sites and/or cleavage sites.

The plasmids of the invention can be transferred into the chosen host cell by well-known methods. Such methods include, for example, the calcium chloride transformation method for E. coli and the calcium phosphate treatment or electroporation methods for mammalian cells. Cells transformed by the plasmids can be selected by resistance to antibiotics conferred by genes contained on the plasmids, such as the amp, gpt, neo, and hyg genes.

Once expressed, the recombinant or naturally occurring ADNF polypeptides can be purified according to standard procedures of the art, including ammonium sulfate precipitation, affinity columns, column chromatography, gel electrophoresis and the like (see, e.g., Scopes, Polypeptide Purification (1982); Deutscher, Methods in Enzymology Vol. 182: Guide to Polypeptide Purification (1990)). Once purified, partially or to homogeneity as desired, the ADNF polypeptides may then be used, e.g., to prevent or treat an autoimmune disease (e.g., multiple sclerosis) in a subject. See, also, e.g., Brenneman & Gozes, J. Clin. Invest. 97:2299-2307 (1996), Brenneman et al., J. Pharm. Exp. Ther. 285:619-627 (1998), and Zamostiano et al., J. Biol. Chem. 276:708-714 (2001), Bassan et al. J. Neurochem 72:1283-1293 (1999), the teachings of which are hereby incorporated in their entirety by reference

B. Synthesis of ADNF Polypeptides

In addition to the foregoing recombinant techniques, the ADNF polypeptides of the invention are optionally synthetically prepared via a wide variety of well-known techniques. Polypeptides of relatively short size are typically synthesized in solution or on a solid support in accordance with conventional techniques (see, e.g., Merrifield, Am. Chem. Soc. 85:2149-2154 (1963)). Various automatic synthesizers and sequencers are commercially available and can be used in accordance with known protocols (see, e.g., Stewart & Young, Solid Phase Peptide Synthesis (2nd ed. 1984)). Solid phase synthesis in which the C-terminal amino acid of the sequence is attached to an insoluble support followed by sequential addition of the remaining amino acids in the sequence is the preferred method for the chemical synthesis of the polypeptides of this invention. Techniques for solid phase synthesis are described by Barany & Merrifield, Solid-Phase Peptide Synthesis; pp. 3-284 in The Peptides: Analysis, Synthesis, Biology. Vol. 2: Special Methods in Peptide Synthesis, Part A.; Merrifield et al., J. Am. Chem. Soc. 85:2149-2156 (1963); and Stewart et al., Solid Phase Peptide Synthesis (2nd ed. 1984).

After chemical synthesis, biological expression or purification, the polypeptide(s) may possess a conformation substantially different than the native conformations of the constituent polypeptides. In this case, it is helpful to denature and reduce the polypeptide and then to cause the polypeptide to re-fold into the preferred conformation. Methods of reducing and denaturing polypeptides and inducing re-folding are well known to those of skill in the art (see Debinski et al., J. Biol. Chem. 268:14065-14070 (1993); Kreitman & Pastan, Bioconjug. Chem. 4:581-585 (1993); and Buchner et al., Anal. Biochem. 205:263-270 (1992)). Debinski et al., for example, describe the denaturation and reduction of inclusion body polypeptides in guanidine-DTE. The polypeptide is then refolded in a redox buffer containing oxidized glutathione and L-arginine.

One of skill will recognize that modifications can be made to the polypeptides without diminishing their biological activity. Some modifications may be made to facilitate the cloning, expression, or incorporation of the targeting molecule into a fusion polypeptide. Such modifications are well known to those of skill in the art and include, for example, a methionine added at the amino terminus to provide an initiation site, or additional amino acids (e.g., poly His) placed on either terminus to create conveniently located restriction sites or termination codons or purification sequences.

C. Conservative Modifications of the ADNF Nucleic Acids and Polypeptides

One of skill will appreciate that many conservative variations of the ADNF nucleic acid and polypeptide sequences provided herein yield functionally identical products. For example, due to the degeneracy of the genetic code, “silent substitutions” (i.e., substitutions of a nucleic acid sequence that do not result in an alteration in an encoded polypeptide) are an implied feature of every nucleic acid sequence that encodes an amino acid. Similarly, “conservative amino acid substitutions,” in one or a few amino acids in an amino acid sequence are substituted with different amino acids with highly similar properties (see the definitions section, supra), are also readily identified as being highly similar to a disclosed amino acid sequence, or to a disclosed nucleic acid sequence that encodes an amino acid. Such conservatively substituted variations of each explicitly listed nucleic acid and amino acid sequences are a feature of the present invention.

One of skill will recognize many ways of generating alterations in a given nucleic acid sequence. Such well-known methods include site-directed mutagenesis, PCR amplification using degenerate oligonucleotides, exposure of cells containing the nucleic acid to mutagenic agents or radiation, chemical synthesis of a desired oligonucleotide (e.g., in conjunction with ligation and/or cloning to generate large nucleic acids) and other well-known techniques (see Giliman & Smith, Gene 8:81-97 (1979); Roberts et al., Nature 328:731-734 (1987)). For example, alanine scanning can be used to determine conservatively modified variants for NAPVSIPQ (i.e., by substituting each amino acid one by one with an alanine or other small neutral amino acid and assay for activity as described herein).

Polypeptide sequences can also be altered by changing the corresponding nucleic acid sequence and expressing the polypeptide. Polypeptide sequences are also optionally generated synthetically using commercially available peptide synthesizers to produce any desired polypeptide (see, Merrifield, supra, and Stewart & Young, supra).

More particularly, it will be readily apparent to those of ordinary skill in the art that the ADNF polypeptides of the present invention can readily be screened for their ability to prevent or treat multiple sclerosis using various assays known in the art or described herein.

Using these assays, one of ordinary skill in the art can readily prepare a large number of ADNF polypeptides in accordance with the teachings of the present invention and, in turn, screen them using the foregoing assay to find ADNF III polypeptides, in addition to those set forth herein, which possess the neuroprotective/neurotrophic activity of the intact ADNF III growth factor. For instance, using ADNF 111-8 (i.e., Asn-Ala-Pro-Val-Ser-Ile-Pro-Gln) as a starting point, one can systematically add, for example, Gly-, Gly-Gly-, Leu-Gly-Gly- to the N-terminus of ADNF III-8 and, in turn, screen each of these ADNF III polypeptides in the foregoing assay to determine whether they possess neuroprotective/neurotrophic activity. In doing so, it will be found that additional amino acids can be added to both the N-terminus and the C-terminus of the newly discovered active site, i.e., Asn-Ala-Pro-Val-Ser-Ile-Pro-Gln, without loss of biological activity as evidenced by the fact that the intact ADNF III growth factor exhibits extraordinary biological activity. This discussion also applies to ADNF I polypeptides.

### EXAMPLE

The following example is offered to illustrate, but not to limit the claimed invention.

The present study focuses on the axonal damage and demyelination in the Myelin-oligodendrocyte glycoprotein (MOG)-induced chronic autoimmune encephalomyelitis (EAE) model. MOG-induced EAE is an accepted animal model of MS. A previous study by Offen and Melamed investigated the possible role of axonal susceptibility and resistance to reactive oxygen species (ROS) in the pathogenesis of EAE. They used neuron-specific-enolase-bcl-2 transgenic mice that over-express the human bcl-2 gene exclusively in their neurons, under the control of the neuron specific enolase promoter. This study demonstrates that the clinical features in MOG-induced EAE, as well as inflammation and axonal damage, are significantly attenuated in NSE-bcl-2 mice (Offen D et al. J Mol Neurosci. 15(3): 167-76 (2000)).

The present study demonstrates that administration of the NAP (Asn-Ala-Pro-Val-Ser-Ile-Pro-Gln, single-letter code: NAPVSIPQ) peptide decreases disease indications in MOG-induced EAE mice.

Methods and Results

EAE was induced by immunization with the peptide encompassing amino acids 35-55 of rat MOG. Synthesis was carried out by the Weizmann Institute Synthesis Unit using a solid-phase technique, on a peptide synthesizer (Applied Biosystems Inc., Foster City, Calif. City). Six weeks old C57/b mice (Tel-Aviv University) were injected (subcutaneous) in the flank with a 2001 μl emulsion containing 300 μg MOG peptide in complete Freund adjuvant (CFA) and 500 μg Mycobacterium tuberculosis (Sigma Israel). An identical booster immunization was given on the other flank one week later. Ten days following the encephalitogenic challenge, the MOG-treated mice were observed daily and the clinical manifestations of EAE were measured by the following score: 0=no clinical symptoms; 1=loss of tail tonicity; 2=partial hind limb paralysis; 3=complete hind limb paralysis; 4=partial frontal limb paralysis; 5=complete frontal limb paralysis; 6=death.

For treatment, NAP was administered (intranasal) 0.1 microgram/mouse in a mixture containing 7.5 mg/ml sodium chloride, 1.7 mg/ml citric acid monohydrate, 3.0 mg/ml disodium phosphate dehydrate and 0.2 mg/ml of a 50% benzalkonium chloride solution. The nasal administration was given daily, 1 hour after MOG injection and was continued and given once a day, 1 hour prior to testing. Control animals received the above mixture without NAP. In the example here, NAP's daily treatment began 10-14 days prior to the MOG injection.

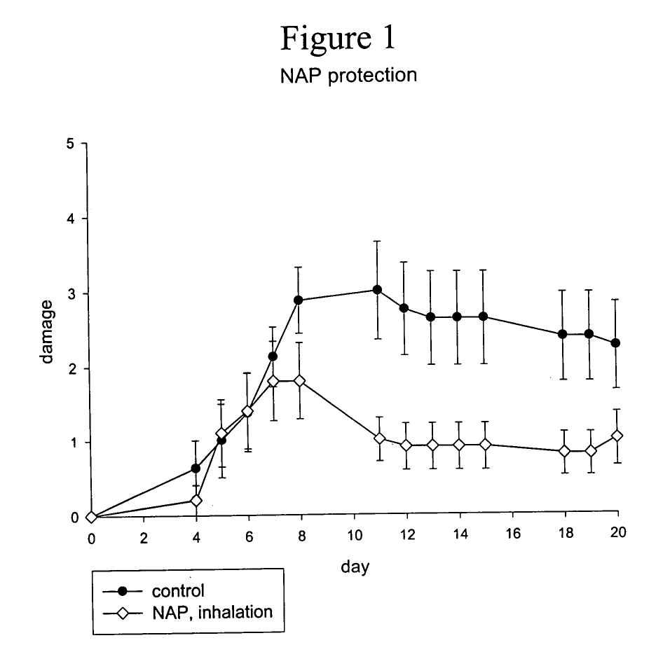

Results showed that NAP significantly improved the clinical outcome of the animals, day 11 on, P<0.01, t-test (FIG. 1).

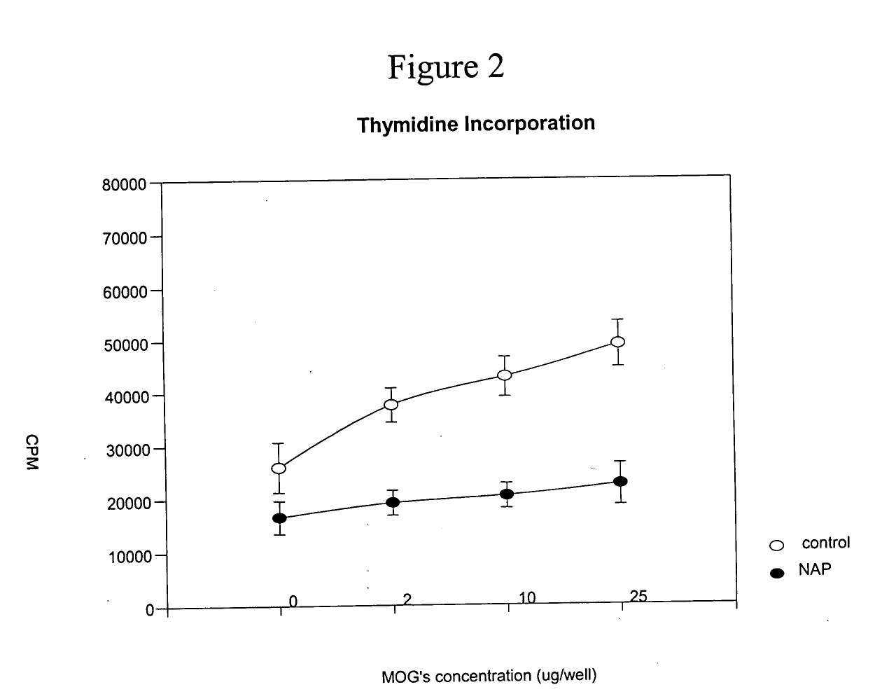

An additional experiment included proliferative T-cell response performed as described by Offen et al., supra. Results indicated that NAP inhibited the immune response (cell proliferation, FIG. 2) in vivo as the proliferative response of splenocytes was much reduced (P<0.01) in the MS model treated with NAP as compared to untreated. Furthermore, addition of MOG resulted in increased proliferation in the untreated splenotcytes (even at 2 micrograms/well of MOG, P<0.05), while in NAP injected animals even at 25 micrograms MOG there was no effect.

While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

All publications and patent documents cited in this application are incorporated by reference in their entirety for all purposes to the same extent as if each individual publication or patent document were so individually denoted. By their citation of various references in this document, Applicants do not admit any particular reference is “prior art” to their invention.

1. A method for preventing or treating an autoimmune disease in a subject, the method comprising the step of administering to the subject a therapeutically effective amount of an Activity Dependent Neurotrophic Factor (ADNF) polypeptide, wherein the ADNF polypeptide is a member selected from the group consisting of:
(a) an ADNF I polypeptide comprising an active core site having the following amino acid sequence:

| | |
|---|---|
| Ser-Ala-Leu-Leu-Arg-Ser-Ile-Pro-Ala; | (SEQ ID NO: 1) |

(b) an ADNF III polypeptide comprising an active core site having the following amino acid sequence:
Asn-Ala-Pro-Val-Ser-Ile-Pro-Gln (SEQ ID NO:2); and
(c) a mixture of the ADNF I polypeptide of part (a) and the ADNF III polypeptide of part (b).

2. The method of claim 1, wherein the ADNF polypeptide is a member selected from the group consisting of a full length ADNF I polypeptide, a full length ADNF III polypeptide, and a mixture of a full length ADNF I polypeptide and a full length ADNF III polypeptide.

3. The method of claim 1, wherein the ADNF polypeptide is an ADNF I polypeptide.

4. The method of claim 3, wherein the active core site of the ADNF I polypeptide comprises at least one D-amino acid.

5. The method of claim 3, wherein the active core site of the ADNF I polypeptide comprises all D-amino acids.

6. The method of claim 3, wherein the ADNF I polypeptide is Ser-Ala-Leu-Leu-Arg-Ser-Ile-Pro-Ala (SEQ ID NO:1).

7. The method of claim 3, wherein the ADNF I polypeptide is selected from the group consisting of:

| | |
|---|---|
| Val-Leu-Gly-Gly-Gly-Ser-Ala-Leu- | (SEQ ID NO: 3) |
| Leu-Arg-Ser-Ile-Pro-Ala; | |
| Val-Glu-Glu-Gly-Ile-Val-Leu-Gly- | (SEQ ID NO: 4) |
| Gly-Gly-Ser-Ala-Leu-Leu-Arg-Ser- | |
| Ile-Pro-Ala; | |
| Leu-Gly-Gly-Gly-Ser-Ala-Leu-Leu- | (SEQ ID NO: 5) |
| Arg-Ser-Ile-Pro-Ala; | |
| Gly-Gly-Gly-Ser-Ala-Leu-Leu-Arg- | (SEQ ID NO: 6) |
| Ser-Ile-Pro-Ala; | |
| Gly-Gly-Ser-Ala-Leu-Leu-Arg-Ser- | (SEQ ID NO: 7) |
| Ile-Pro-Ala; | |
| Gly-Ser-Ala-Leu-Leu-Arg-Ser-Ile- | (SEQ ID NO: 8) |
| Pro-Ala; and | |
| Ser-Ala-Leu-Leu-Arg-Ser-Ile-Pro- | (SEQ ID NO: 1) |
| Ala. | |

8. The method of claim 3, wherein the ADNF I polypeptide comprises up to about 20 amino acids at at least one of the N-terminus and the C-terminus of the active core site.

9. The method of claim 1, wherein the ADNF polypeptide is an ADNF III polypeptide.

10. The method of claim 9, wherein the ADNF polypeptide is a full length ADNF III polypeptide.

11. The method of claim 9, wherein the ADNF III polypeptide is Asn-Ala-Pro-Val-Ser-Ile-Pro-Gln (SEQ ID NO:2).

12. The method of claim 9, wherein the active core site of the ADNF III polypeptide comprises at least one D-amino acid.

13. The method of claim 9, wherein the active core site of the ADNF III polypeptide comprises all D-amino acids.

14. The method of claim 9, wherein the ADNF III polypeptide is a member selected from the group consisting of:

| | |
|---|---|
| Gly-Gly-Asn-Ala-Pro-Val-Ser-Ile- | (SEQ ID NO: 9) |
| Pro-Gln; | |
| Leu-Gly-Gly-Asn-Ala-Pro-Val-Ser- | (SEQ ID NO: 10) |
| Ile-Pro-Gln-Gln-Ser; | |
| Leu-Gly-Leu-Gly-Gly-Asn-Ala-Pro- | (SEQ ID NO: 11) |
| Val-Ser-Ile-Pro-Gln-Gln-Ser; | |
| Ser-Val-Arg-Leu-Gly-Leu-Gly-Gly- | (SEQ ID NO: 12) |
| Asn-Ala-Pro-Val-Ser-Ile-Pro-Gln- | |
| Gln-Ser; and | |
| Asn-Ala-Pro-Val-Ser-Ile-Pro-Gln. | (SEQ ID NO: 2) |

15. The method of claim 9, wherein the ADNF III polypeptide comprises up to about 20 amino acids at at least one of the N-terminus and the C-terminus of the active core site.

16. The method of claim 1, wherein at least one of the ADNF polypeptides is encoded by a nucleic acid that is administered to the subject.

17. The method of claim 1, wherein an ADNF I polypeptide of part (a) and an ADNF III polypeptide of part (b) are administered to the subject.

18. The method of claim 17, wherein either or both active core sites of the ADNF I polypeptide and the ADNF III polypeptide comprise at least one D-amino acid.

19. The method of claim 17, wherein either or both active core sites of the ADNF I polypeptide and the ADNF III polypeptide comprise all D-amino acids.

20. The method of claim 17, wherein the ADNF I polypeptide is Ser-Ala-Leu-Leu-Arg-Ser-Ile-Pro-Ala (SEQ ID NO:1), and wherein the ADNF III polypeptide is Asn-Ala-Pro-Val-Ser-Ile-Pro-Gln (SEQ ID NO:2).

21. The method of claim 17, wherein the ADNF I polypeptide is a member selected from the group consisting of:

| | |
|---|---|
| Val-Leu-Gly-Gly-Gly-Ser-Ala-Leu-Leu- | (SEQ ID NO: 3) |
| Arg-Ser-Ile-Pro-Ala; | |
| Val-Glu-Glu-Gly-Ile-Val-Leu-Gly-Gly- | (SEQ ID NO: 4) |
| Gly-Ser-Ala-Leu-Leu-Arg-Ser-Ile-Pro- | |
| Ala; | |
| Leu-Gly-Gly-Gly-Ser-Ala-Leu-Leu-Arg- | (SEQ ID NO: 5) |
| Ser-Ile-Pro-Ala; | |
| Gly-Gly-Gly-Ser-Ala-Leu-Leu-Arg-Ser- | (SEQ ID NO: 6) |
| Ile-Pro-Ala; | |
| Gly-Gly-Ser-Ala-Leu-Leu-Arg-Ser-Ile- | (SEQ ID NO: 7) |
| Pro-Ala; | |
| Gly-Ser-Ala-Leu-Leu-Arg-Ser-Ile-Pro- | (SEQ ID NO: 8) |
| Ala; and | |
| Ser-Ala-Leu-Leu-Arg-Ser-Ile-Pro-Ala; | (SEQ ID NO: 1) |
| and | |

wherein the ADNF III polypeptide is selected from the group consisting of:

| | |
|---|---|
| Gly-Gly-Asn-Ala-Pro-Val-Ser-Ile- | (SEQ ID NO: 9) |
| Pro-Gln; | |
| Leu-Gly-Gly-Asn-Ala-Pro-Val-Ser- | (SEQ ID NO: 10) |
| Ile-Pro-Gln-Gln-Ser; | |
| Leu-Gly-Leu-Gly-Gly-Asn-Ala-Pro- | (SEQ ID NO: 11) |
| Val-Ser-Ile-Pro-Gln-Gln-Ser; | |
| Ser-Val-Arg-Leu-Gly-Leu-Gly-Gly- | (SEQ ID NO: 12) |
| Asn-Ala-Pro-Val-Ser-Ile-Pro-Gln- | |
| Gln-Ser; and | |
| Asn-Ala-Pro-Val-Ser-Ile-Pro-Gln. | (SEQ ID NO: 2) |

22. The method of claim 17, wherein the ADNF I polypeptide comprises up to about 20 amino acids at at least one of the N-terminus and the C-terminus of the active core site of the ADNF I polypeptide, and wherein the ADNF III polypeptide comprises up to about 20 amino acids at at least one of the N-terminus and the C-terminus of the active core site of the ADNF III polypeptide.

23. The method of claim 1, wherein the subject has an autoimmune disease.

24. The method of claim 1, wherein the ADNF polypeptide is administered to prevent an autoimmune disease.

25. The method of claim 1, wherein the autoimmune disease is selected from the group consisting of multiple sclerosis, myasthenia gravis, Guillan-Barre syndrome (antiphospholipid syndrome), systemic lupus erytromatosis, Behcet's syndrome, Sjogrens syndrome, rheumatoid arthritis, Hashimoto's disease/hypothyroiditis, primary biliary cirrhosis, mixed connective tissue disease, chronic active hepatitis, Graves' disease/hyperthyroiditis, scleroderma, chronic idiopathic thrombocytopenic purpura, diabetic neuropathy and septic shock.

26. The method of claim 1, wherein the ADNF polypeptide is administered intranasally.

27. The method of claim 1, wherein the ADNF polypeptide is administered orally.

28. The method of claim 1, wherein the ADNF polypeptide is injected.


SEQUENCE LISTING (23 sequences)

SEQ ID NO: 1 - protein, 9 aa, Artificial Sequence
  Description of Artificial Sequenceactivity dependent neurotrophic factor I (ADNF I) active core site (SAL, ADNF-9)
SALLRSIPA

SEQ ID NO: 2 - protein, 8 aa, Artificial Sequence
  Description of Artificial Sequenceactivity dependent neurotrophic factor III (ADNF III) active core site (NAP)
NAPVSIPQ

SEQ ID NO: 3 - protein, 14 aa, Artificial Sequence
  Description of Artificial SequenceADNF I polypeptide
VLGGGSALLRSIPA

SEQ ID NO: 4 - protein, 19 aa, Artificial Sequence
  Description of Artificial SequenceADNF I polypeptide
VEEGIVLGGGSALLRSIPA

SEQ ID NO: 5 - protein, 13 aa, Artificial Sequence
  Description of Artificial SequenceADNF I polypeptide
LGGGSALLRSIPA

SEQ ID NO: 6 - protein, 12 aa, Artificial Sequence
  Description of Artificial SequenceADNF I polypeptide
GGGSALLRSIPA

SEQ ID NO: 7 - protein, 11 aa, Artificial Sequence
  Description of Artificial SequenceADNF I polypeptide
GGSALLRSIPA

SEQ ID NO: 8 - protein, 10 aa, Artificial Sequence
  Description of Artificial SequenceADNF I polypeptide
GSALLRSIPA

SEQ ID NO: 9 - protein, 10 aa, Artificial Sequence
  Description of Artificial SequenceADNF III polypeptide
GGNAPVSIPQ

SEQ ID NO: 10 - protein, 13 aa, Artificial Sequence
  Description of Artificial SequenceADNF III polypeptide
LGGNAPVSIPQQS

SEQ ID NO: 11 - protein, 15 aa, Artificial Sequence
  Description of Artificial SequenceADNF III polypeptide
LGLGGNAPVSIPQQS

SEQ ID NO: 12 - protein, 18 aa, Artificial Sequence
  Description of Artificial SequenceADNF III polypeptide
SVRLGLGGNAPVSIPQQS

SEQ ID NO: 13 - protein, 89 aa, Artificial Sequence
  Description of Artificial SequenceADNF I polypeptide
XXXXXXXXXXXXXXXXXXXXXXXXXXXXXXXXXXXXXXXXSALLRSIPAXXXXXXXXXXXXXXXXXXXXXXXXXXXXXXXXXXXXXXXX

SEQ ID NO: 14 - protein, 88 aa, Artificial Sequence
  Description of Artificial SequenceADNF III polypeptide
XXXXXXXXXXXXXXXXXXXXXXXXXXXXXXXXXXXXXXXXNAPVSIPQXXXXXXXXXXXXXXXXXXXXXXXXXXXXXXXXXXXXXXXX

SEQ ID NO: 15 - protein, 5 aa, Artificial Sequence
  Description of Artificial Sequence1-R or 2-R within the formula for ADNF I polypeptide
VLGGG

SEQ ID NO: 16 - protein, 4 aa, Artificial Sequence
  Description of Artificial Sequence2-R within the formula for ADNF I polypeptide
VLGG

SEQ ID NO: 17 - protein, 5 aa, Artificial Sequence
  Description of Artificial Sequence2-R within the formula for ADNF I polypeptide
VLGGV

SEQ ID NO: 18 - protein, 5 aa, Artificial Sequence
  Description of Artificial Sequence2-R within the formula for ADNF I polypeptide
GVLGG

SEQ ID NO: 19 - protein, 5 aa, Artificial Sequence
  Description of Artificial Sequence3-R or 4-R within the formula for ADNF III polypeptide
LGLGG

SEQ ID NO: 20 - protein, 4 aa, Artificial Sequence
  Description of Artificial Sequence4-R within the formula for ADNF III polypeptide
LGLG

SEQ ID NO: 21 - protein, 5 aa, Artificial Sequence
  Description of Artificial Sequence4-R within the formula for ADNF III polypeptide
LGLGL

SEQ ID NO: 22 - protein, 10 aa, Artificial Sequence
  Description of Artificial Sequence1-R within the formula for ADNF I polypeptide
VEEGIVLGGG

SEQ ID NO: 23 - protein, 8 aa, Artificial Sequence
  Description of Artificial Sequence3-R within the formula for ADNF III polypeptide
SVRLGLGG